(19) United States
(12) Patent Application Publication
(10) Pub. No.: US 2008/0097319 A1
(43) Pub. Date: Apr. 24, 2008

(54) Active squeezing device for a drip bag

(75) Inventors: Tzu-Cheng Shih, Taipei (TW); Chin-Liang Lin, Taipei (TW); Kuan-Yii Chen, Taipei (TW); Shail-Chen Yu, Taipei (TW); Chorng-Shyan Lin, Taoyuan Hsien (TW)

Correspondence Address: BIRCH STEWART KOLASCH & BIRCH, PO BOX 747, FALLS CHURCH, VA, 22040-0747, US

(73) Assignee: NATIONAL DEFENSE MEDICAL CENTER

(21) Appl. No.: 11/522,329

(22) Filed: Sep. 18, 2006

(51) Int. Cl. A61M 5/142; A61M 37/00

(52) U.S. Cl. 604/131

(57) ABSTRACT

An active squeezing device is used with a drip bag has a drip bag holder and a magnetic pressure assembly. The drip bag holder has a base and a cover mounted pivotally on the base. The magnetic pressure assembly is mounted on the drip bag holder and selectively generates an attractive magnetic force to tightly close the cover on the base to hold and squeeze the drip bag between the base and the cover or generates a repulsive magnetic force to open the cover to release the drip bag. The active squeezing device uses the magnetic force to actively transfuse the liquid medicine or blood in the drip bag to a patient without hanging the drip bag over the patient.

### BACKGROUND OF THE INVENTION

1. Field of the Invention

The present invention relates to a squeezing device, and more particularly to an active squeezing device that is used with a drip bag, is portable, holds and actively squeezes the drip bag to transfuse liquid medicine or blood in the drip bag to a patient.

2. Description of Related Art

Medical drips are used to store and transfuse blood or liquid medicine to a patient and have a drip bag, a pipe and a needle. The drip bag stores the liquid medicine or blood. The pipe is mounted and communicates with the drip bag and has a distal end. The needle is mounted on the distal end and communicates with the pipe. A drip is used generally with a stand holding the drip bag over a patient so that the liquid medicine or blood in the drip bag flows into the patient's body due to the pressure difference of the liquid medicine or blood and the patient's blood.

However, carrying the stand with the patient is inconvenient and even impossible when the patent moves to a narrow and small space such as an internal cavity of a hyperbaric oxygen therapy capsule due to the narrow and small space having insufficient height to uprightly set the stand. The stand cannot maintain an adequate pressure difference between the liquid medicine or blood and the patient's blood without being set uprightly.

Moreover, the conventional stand is made of metal, is heavy and over burdens a weak patient when the patient moves and carries the stand. Although the patient may be accompanied with a care worker who carries the stand and the drip, carrying and moving the heavy stand on a slope or stairs is laborious and inconvenient.

With reference to FIG. 14, Taiwan patent No. M281637 discloses a drip holder used for holding a drip and comprising a back bracket (90), two lower tubes (91), two upper rods (92) and two hangers (93). The back bracket (90) has two shoulder straps to allow a patient to shoulder the back bracket (90). The lower tubes (91) are mounted retractably in the back bracket (90). The upper rods (92) are retractably mounted respectively in the lower tubes (91) and each upper rod (92) has a top end. The hangers (93) are formed respectively on the top ends of the upper rods (92) to hang the drip on. The drip holder is light and compact as compared with a conventional metal drip stand and the patient may carry the drip conveniently through the drip holder. However, the drip holder has to hang a drip bag of the drip over the patient in order to transfuse the liquid medicine or blood in the drip bag to the patient's body. Furthermore, the patient cannot shoulder the drip holder when the patient lies. Therefore, the drip holder still cannot be applied to a small space such as an internal cavity of a hyperbaric oxygen therapy capsule without a sufficient height.

With reference to FIG. 15, an improved drip has been developed to overcome the defect of conventional drips that has to be hung over a patient. The improved drip has a bottle (80), a drip bag (81) and a pipe (82). The drip bag (81) is made of resilient material, is similar to a balloon, is put in the bottle (80) and contains liquid medicine or blood. The pipe (82) is connected to and communicates with the drip bag (81). When the improved drip transfuses the liquid medicine or blood to a patient, the drip bag (81) squeezes the liquid medicine or blood out with a contracting force due to its resilience. The improved drip actively squeezing the liquid medicine or blood without hanging over a patient may be used in a small space despite a height of the small space. However, the contracting force of the resilient drip bag (81) is limited and therefore the capacity of the drip bag (81) is insufficient.

To overcome the shortcomings, the present invention provides an active squeezing device for a drip bag to mitigate or obviate the aforementioned problems.

### SUMMARY OF THE INVENTION

The main objective of the invention is to provide an active squeezing device that is used with a drip bag, is portable, holds and actively squeezes the drip bag to transfuse liquid medicine or blood in the drip bag to a patient.

The active squeezing device in accordance with the present invention is used with a drip bag has a drip bag holder and a magnetic pressure assembly. The drip bag holder has a base and a cover mounted pivotally on the base. The magnetic pressure assembly is mounted on the drip bag holder and selectively generates an attractive magnetic force to tightly close the cover on the base to hold and squeeze the drip bag between the base and the cover or generates a repulsive magnetic force to open the cover to release the drip bag.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of an active squeezing device for a drip bag in accordance with the present invention;

FIG. 2 is an exploded and enlarged perspective view of the cover of the drip bag holder and the magnetic pressure assembly in FIG. 1;

FIG. 2A is an exploded and enlarged perspective view of the cover and base of the drip bag holder and the magnetic pressure assembly in FIG. 1;

FIG. 3 is a perspective view of the active squeezing device in FIG. 1 with the top panel of the cover of the drip bag holder being removed;

FIG. 4 is an operational perspective view of the active squeezing device in FIG. 1 with the switch handle being rotated 90 degrees to generate a repulsive magnetic force between the cover and the base of the drip bag holder to open the cover and receive the drag bag;

FIG. 5 is an operational perspective view of the active squeezing device in FIG. 4 with a the switch handle being reversely rotated 90 degrees, being folded and lying on the cover to generate an attractive magnetic force between the cover and the base to tightly clip and squeeze the drip bag;

FIG. 6 is a partially exploded perspective view of the active squeezing device in FIG. 1 with the limits;

FIG. 7 is a perspective view of the active squeezing device in FIG. 7 with the cover being opened;

FIG. 8 is an operational perspective view of the active squeezing device in FIG. 6 clipping the drip bag with one limit mounted on the base close to the proximal end to prevent the cover from further pivoting;

FIG. 9 is an operational perspective view of the active squeezing device in FIG. 6 clipping the drip bag with one limit mounted on the base close to the middle to prevent the cover from further pivoting;

FIG. 10 is an operational perspective view of the active squeezing device in FIG. 6 clipping the drip bag with one limit mounted on the base close to the distal end to prevent the cover from further pivoting;

FIG. 11 is a perspective view of the active squeezing device in FIG. 1 further having an auxiliary pressure assembly to provide an additional squeezing force to the drip bag;

FIG. 12 is a perspective view of the active squeezing device in FIG. 1 further having an elastic band to provide an additional squeezing force to the drip bag;

FIG. 13 is a perspective view of the active squeezing device in FIG. 1 used with a blood pressure cuff and an air pump to provide a further squeezing force to the drip bag;

FIG. 14 is an operational perspective view of a conventional drip bag holder in accordance with the prior art hanging a drip and shouldered by a patient; and

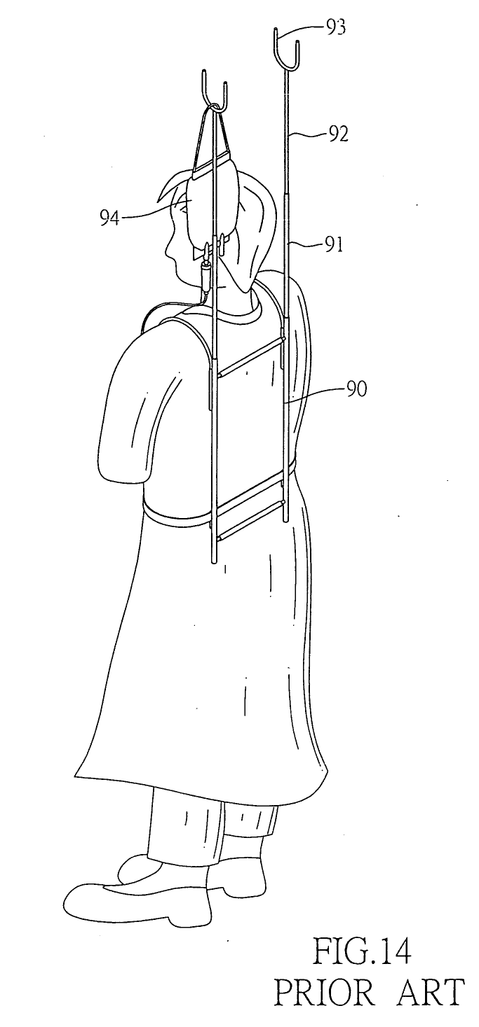

FIG. 15 is a cross sectional side view of a conventional drip with a resilient drip bag in accordance with the prior art.

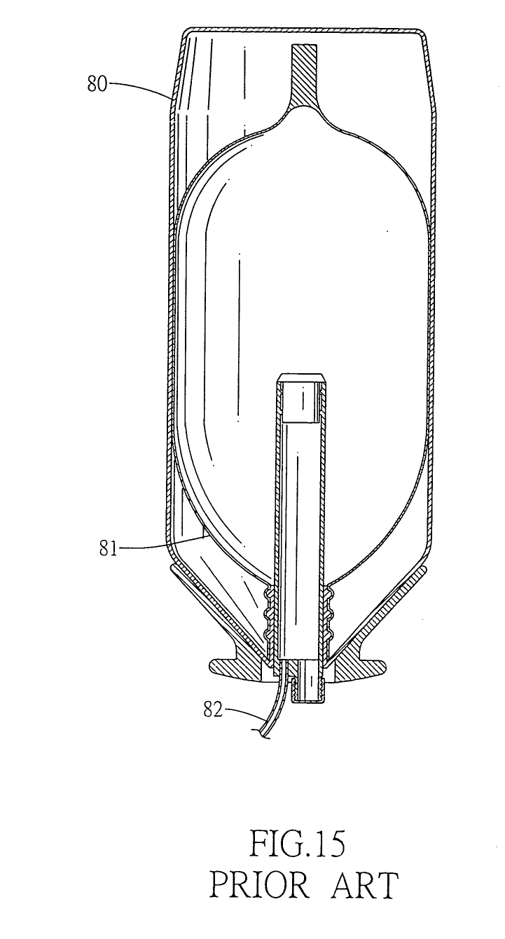

### DETAILED DESCRIPTION OF THE PREFERRED EMBODIMENT

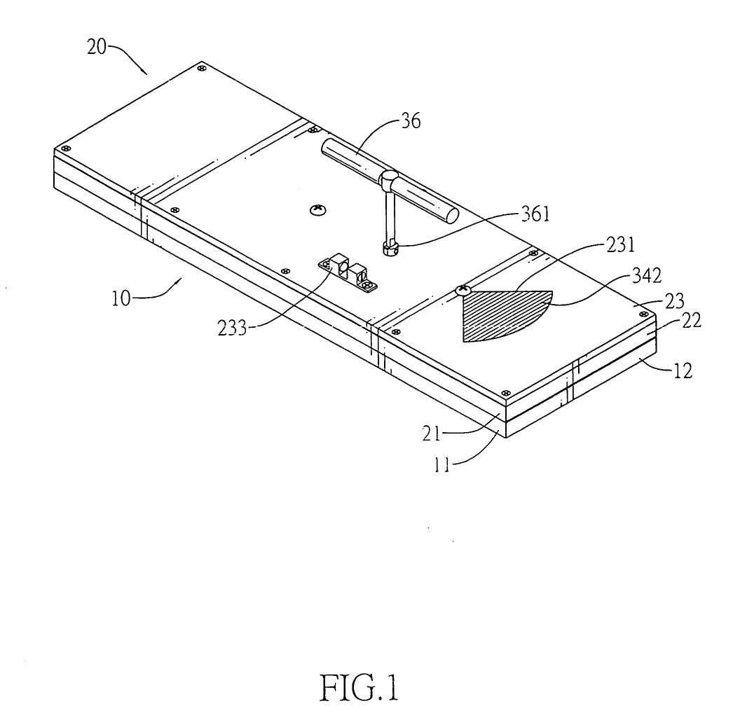

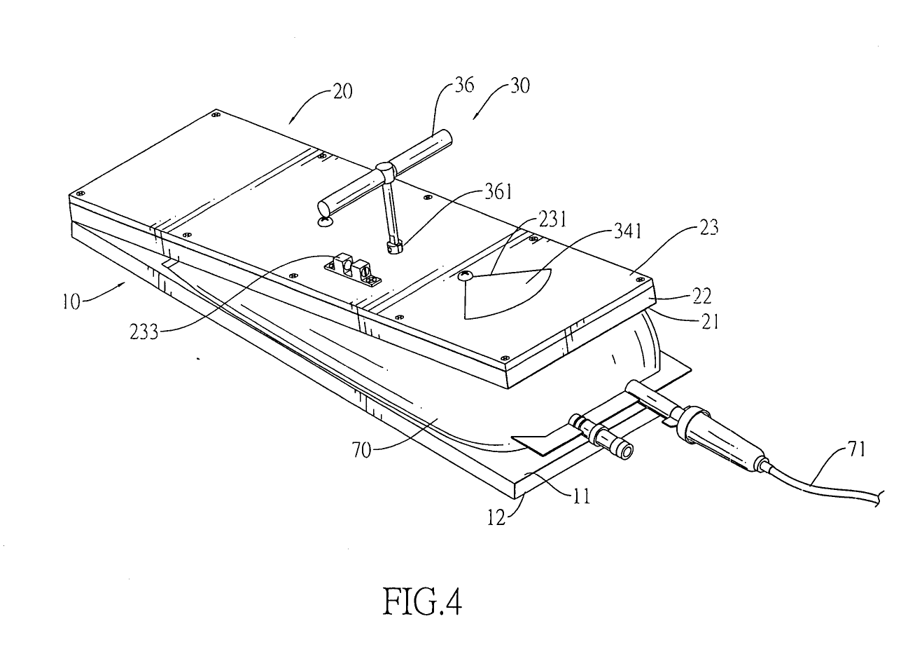

With reference to FIGS. 1 and 4, an active squeezing device in accordance with the present invention is used to hold a drip bag (70) and transfuse liquid medicine or blood in the drip bag (70) to a patient. The drip bag (70) is connected to and communicates a pipe (71).

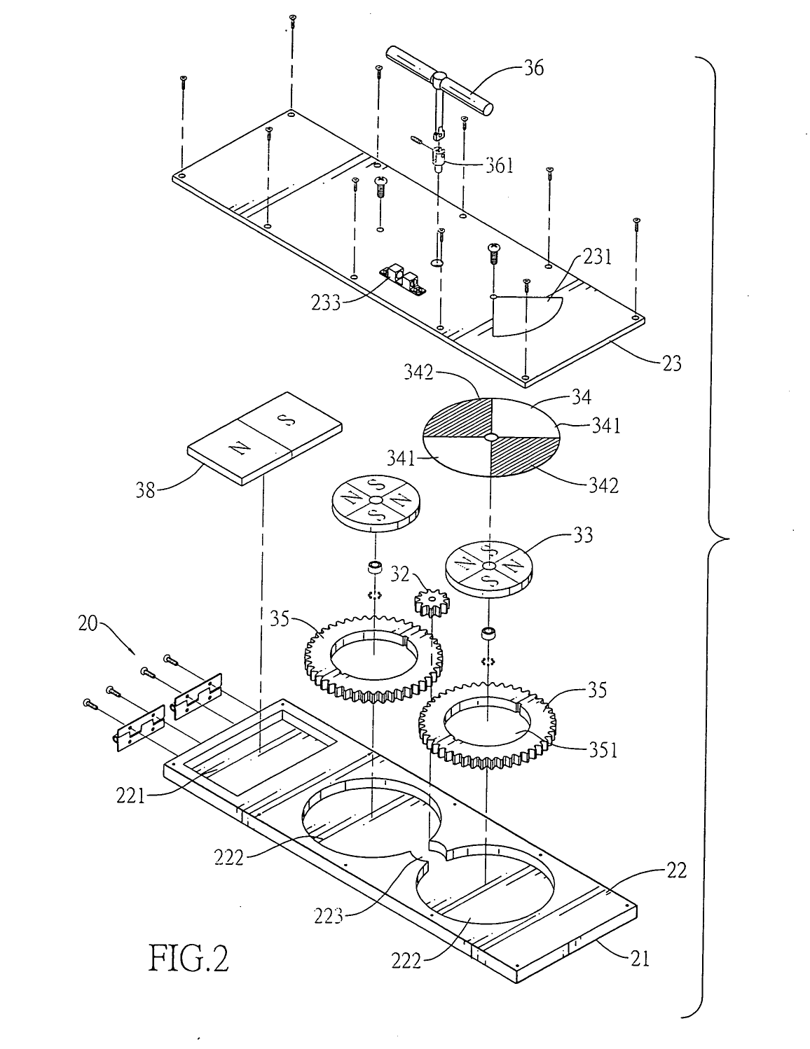

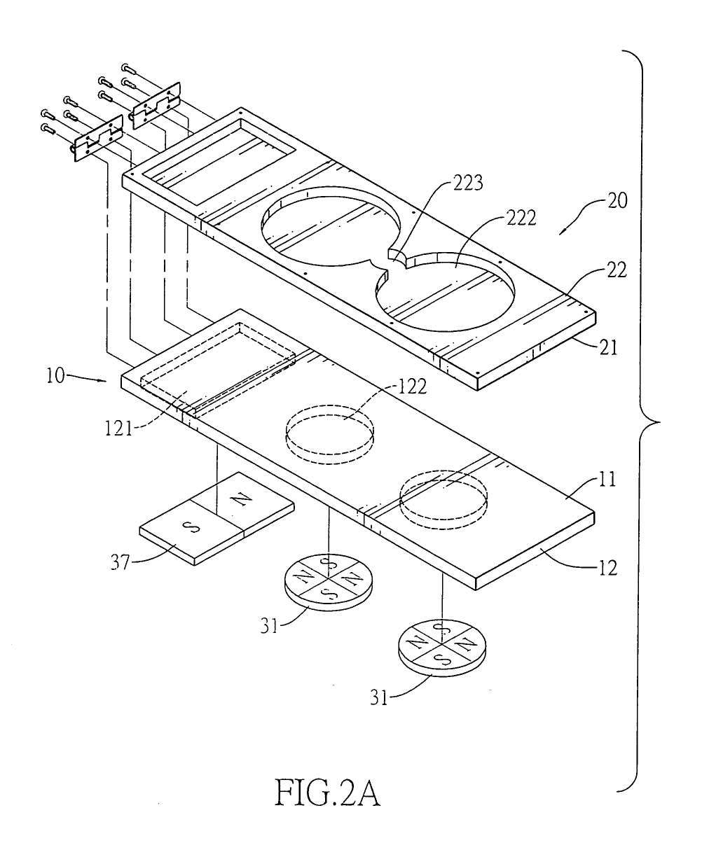

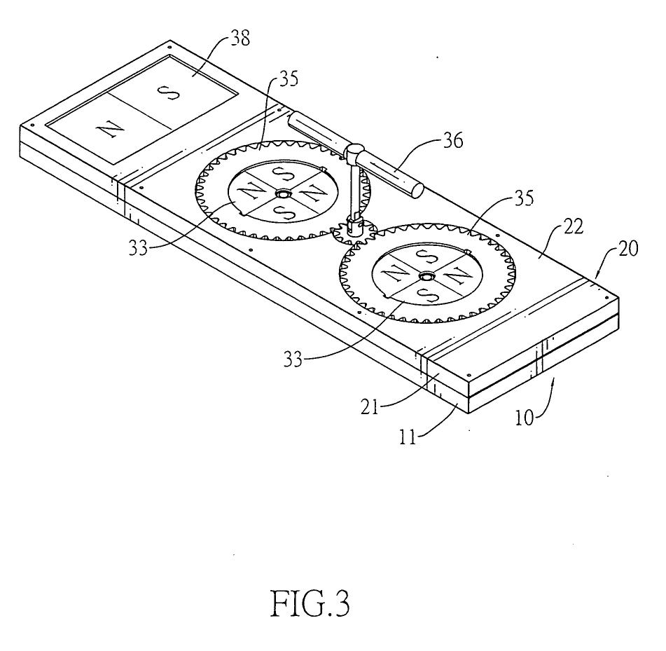

With further reference to FIGS. 2 and 3, the active squeezing device comprises a drip bag holder and a magnetic pressure assembly (30).

With further reference to FIG. 2A, the drip bag holder is portable and has a base (10) and a cover (20).

The base (10) has a proximal end, a distal end, a base board (12) and a metal sheet (11). The base board (12) has a top surface, a bottom surface, two sides, a second recess (121) and at least one first recess (122). The second recess (121) is defined in the bottom surface near the proximal end of the base (10). The at least one first recess (122) is defined in the bottom surface. The metal sheet (11) is mounted on the top surface of the base board (12).

With reference to FIGS. 2 and 2A, the cover (20) is mounted pivotally on the base (10) and has a proximal end, a distal end, a cover board (22), a metal sheet (21) and a top panel (23). The proximal end of the cover (20) is mounted pivotally on the proximal end of the base (10). The cover board (22) is mounted pivotally on the base board (12) and has a top surface, a bottom surface, a second recess (221), at least one first recess (222) and an intermediate recess (223). The second recess (221) is defined in the top surface of the cover board (22) near the proximal end of the cover (20). The at least one first recess (222) is defined in the top surface of the cover board (22). The intermediate recess (223) is defined in the top surface of the cover base (22) and communicates with the at least one first recess (222) in the cover (20). The metal sheet (21) is mounted on the bottom surface of the cover board (22). The top panel (23) is mounted on the top surface of the cover board (22) and has a top surface, a window (231) and a positioning assembly (233). The window (231) is attached to the top panel (23), may be transparent and may be hollow and be defined through the top panel (23). The positioning assembly (233) is mounted on the top surface of the top panel (23) and has two brackets, two retractable balls and two biasing elements. The brackets are mounted on the top surface of the top panel (23) at an interval and each bracket has an inside wall. The retractable balls are retractably mounted respectively in the inside walls of the brackets and face each other. The biasing elements are mounted respectively in the brackets and push respectively the retractable balls partially out of the brackets.

The magnetic pressure assembly (30) is mounted on the drip bag holder and selectively generates an attractive magnetic force to tightly close the cover (20) on the base (10) to hold and squeeze the drip bag (70) or generates a repulsive magnetic force to open the cover (20) to release the drip bag (70). The magnetic pressure assembly has two magnetic elements (37), at least one stationary magnetic disk (31), at least one rotatable magnetic disk (33), a colored disk (34), at least one follower gear (35), a driving gear (32) and a switch handle (36).

The magnetic elements (37, 38) are mounted respectively in the second recesses (121, 221) in the base board (12) of the base (10) and the cover board (22) of the cover (20) and each magnetic element (37, 38) has a south pole (S) and a north pole (N). Different poles of the south and north poles (S, N) of the magnetic elements (37, 38) are aligned with and attract each other.

The at least one stationary magnetic disk (31) is mounted respectively in the at least one first recess (122) in the base board (12) of the base (10) and each of the at least one stationary magnetic disk (31) has multiple south poles (S) and multiple north poles (N). The south and north poles (S, N) are formed radially on the at least one stationary magnetic disk (31) and are arranged alternately.

The at least one rotatable magnetic disk (33) are rotatably mounted respectively in the at least one first recess (222) in the cover board (22) of the cover (20), corresponds to and is aligned respectively with the at least one stationary magnetic disk (31). Each of the at least one rotatable magnetic disk (33) has multiple south poles (S) and north poles (N). The south and north poles (S, N) are formed radially on the at least one rotatable magnetic disk (33) and are arranged alternately. When the at least rotatable magnetic disk (33) rotates to align the south poles (S) of the at least one of the stationary magnetic disk (31) respectively with the north poles (N) of the at least one rotatable disk (33) to generate an attractive magnetic force, the cover (20) is tightly closed on the base (10) by the attractive magnetic force. The closed cover (20) can clip and squeeze the drip bag (70) with the attractive magnetic force. When the at least one rotatable magnetic disk (33) rotates to align the south poles (S) of the at least one stationary magnetic disk (31) respectively with the south poles (S) of the at least one rotatable magnetic disk (31, 33) to generate a repulsive magnetic force, the cover (20) is opened by the repulsive magnetic force to release the drip bag (70).

The colored disk (34) is coaxially mounted on one of the at least one rotatable magnetic disk (33), corresponds to the window (231) on the cover (20) and has multiple first color segments (341) and second color segments (342). The first and second color segments (341, 342) are formed radially on the at least colored disk (34), are arranged alternately and correspond to the south and north poles (S, N) of a corresponding rotatable magnetic disk (34). One of the first and color segments (314, 341) is aligned with the window (231) is revealed through the window (231) to sign a user the alignment or misalignment of the poles (S, N) of the stationary and rotatable magnetic disks (31, 33).

The at least one follower gear (35) is annular and is tightly mounted respectively around the at least one rotatable magnetic disk (33) in the at least first recess (222) in the cover (20). Each of the at least one follower gear (35) has an inner cavity (351) receiving a corresponding rotatable magnetic disk (33).

The driving gear (32) is mounted rotatably in the intermediate recess (223) and engages with the at least one follower gear (35).

A preferred embodiment of the active squeezing device has the base (10) with two first recesses (122), the cover (20) with two first recesses (222) and the intermediate recess (223) defined between the first recesses (222) in the cover (20).

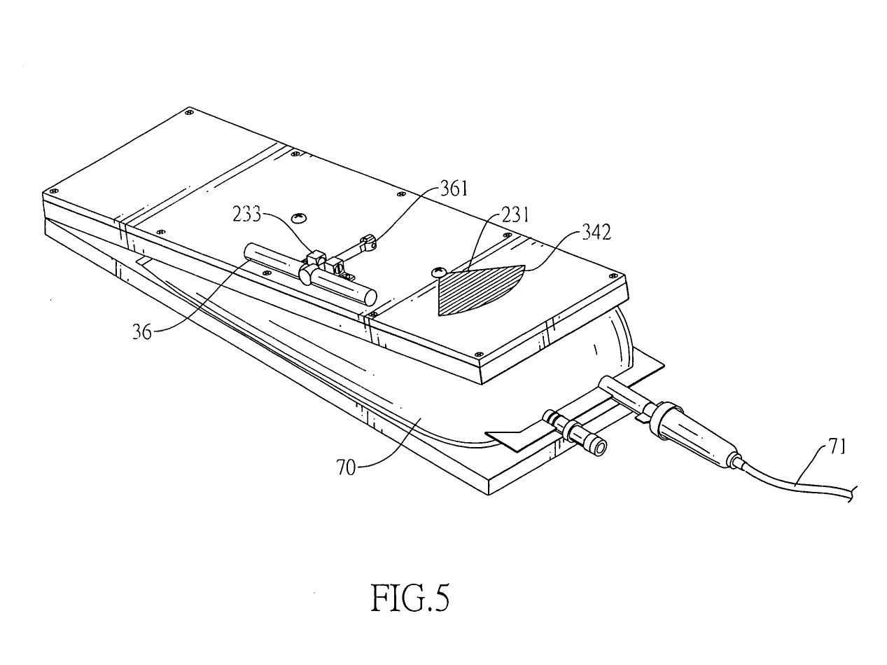

With reference FIGS. 3, 4 and 5, the switch handle (36) is mounted pivotally and rotatably on the cover (20), is connected to and is capable of rotating the at least one rotatable magnetic disk (20) and has a pivot member (361) and a lever. The pivot member (361) is mounted rotatably on the cover (20) through the top panel (23) and is mounted securely on the driving gear (32). The lever is T-shaped and is mounted pivotally on the pivot member (361). The driving gear (32) may be rotated to drive the at least one follower gear (35) to align or misalign the south and north poles (S, N) of the at least one rotatable magnetic disk (33) with those of the at least one stationary magnetic disk (31) by rotating the lever standing on the cover (20). When same poles of the south and north poles (S, N) of the at least one rotatable magnetic disk (33) and the at least one stationary rotatable magnetic disk (31) are misaligned with each other, an attractive magnetic force squeezes the drip bag (70) and transfuses the liquid medicine or blood in the drip bag (70), as shown in FIG. 5. The lever is pivoted to lie on the cover (20) and the positioning assembly (233) positions the lever by blocking the lever between the brackets with the retractable balls to prevent the lever from pivoting inadvertently.

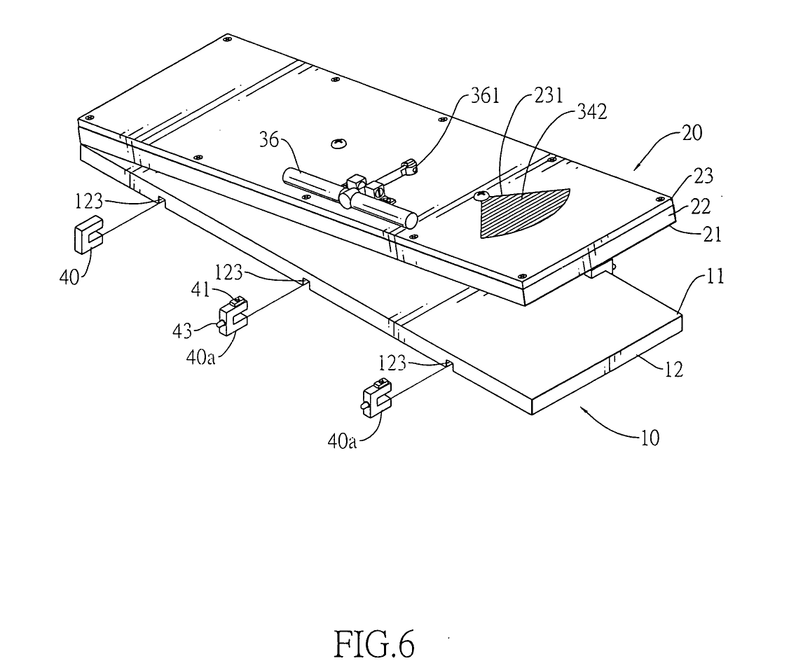

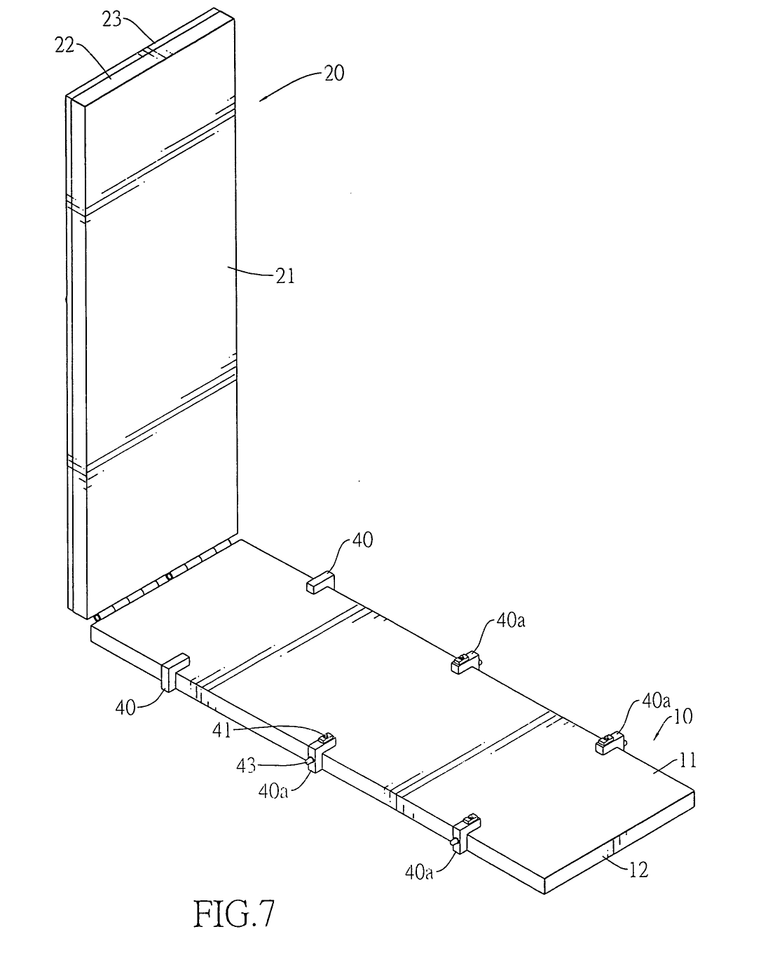

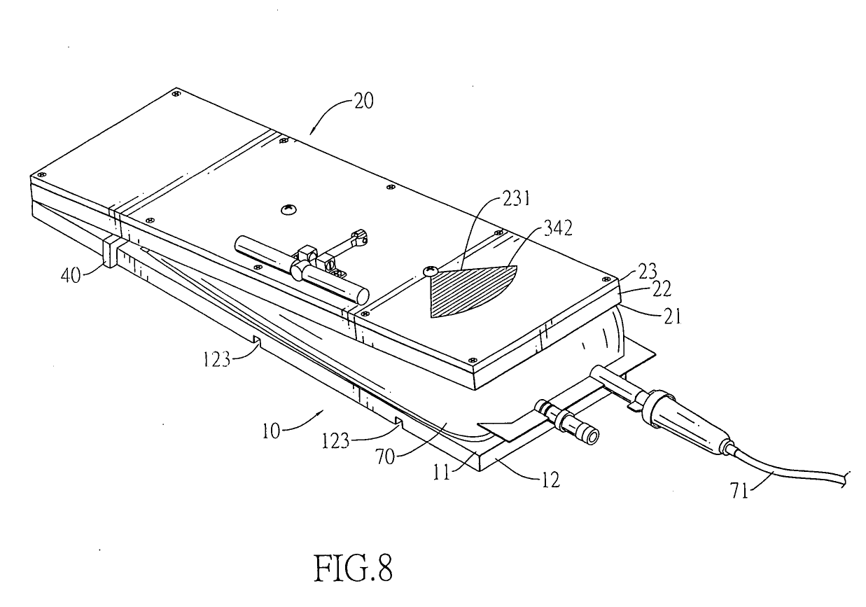

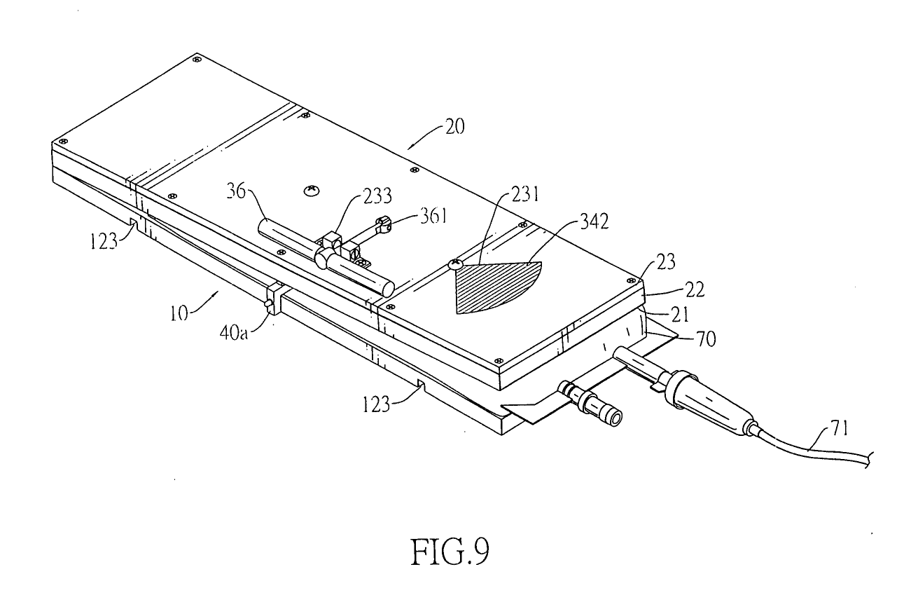

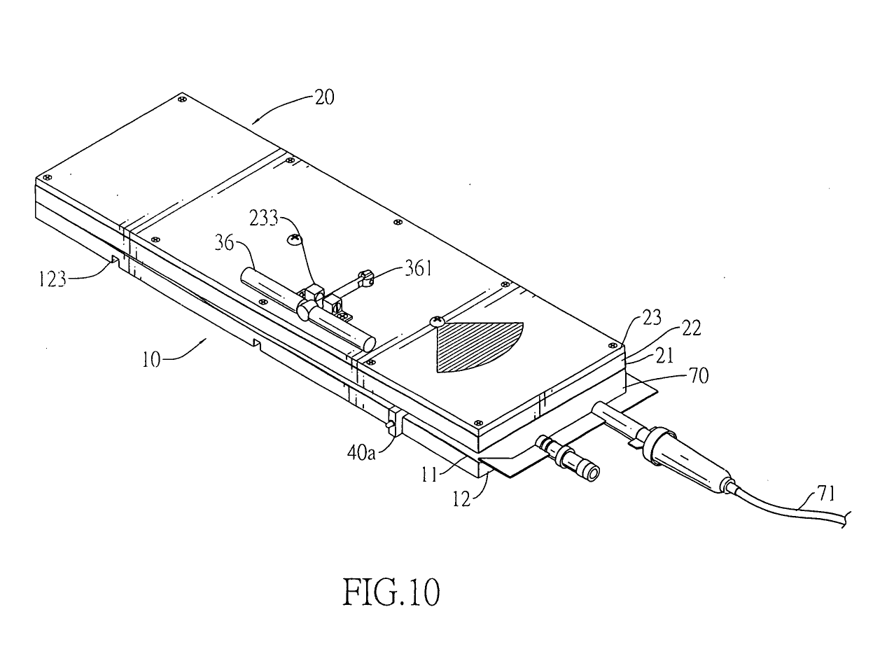

With reference to FIGS. 6-10, a preferred embodiment of the active squeezing device further has the base (10) having two sets of multiple slots (123) and two sets of multiple limits (40, 40a). The sets of the slots (123) are defined respectively in the sides of the base board (12). The slots (123) of each set are arranged longitudinally and respectively represent different scales such as 1000 milliliter (ml), 500 ml and 0 ml to show how many liquid medicine or blood are remaining in or transfused out of the drip bag (70) in the active squeezing holder. Each slot (123) may have a degree number marked on a position on a corresponding side to clearly indicate the degree. The sets of the limits (40, 40a) are mounted respectively on two sides of the base board (12). The limits (40, 40a) of each set are U-shaped, are detachably mounted respectively in the slots (123) in a corresponding side, protrude up from the top surface of the base board (12) to block and prevent the cover (120) from pivoting toward the base (10). The user may transfuse the liquid medicine or blood out of the drip bag (70) step by step by taking off the limits (40, 40a) in turns from the proximal end to the distal end of the base (10). Furthermore, each limit (40a) may have a button (41) and an alarm (43). The button (41) is mounted on the limit (40a) and faces the cover (20) and the cover (20) may pivot down to push the button (41). The alarm (43) may be a light emitting diode (LED) or a buzzer, is mounted on the limit (40a), is connected to the button (41) and is switched by the button (41) when the button (41) is pushed by the cover (20). The buttoned alarm (43) radiates or sounds to notice the user that the liquid medicine or blood has been transfused a quantity as a corresponding slot (123) indicates.

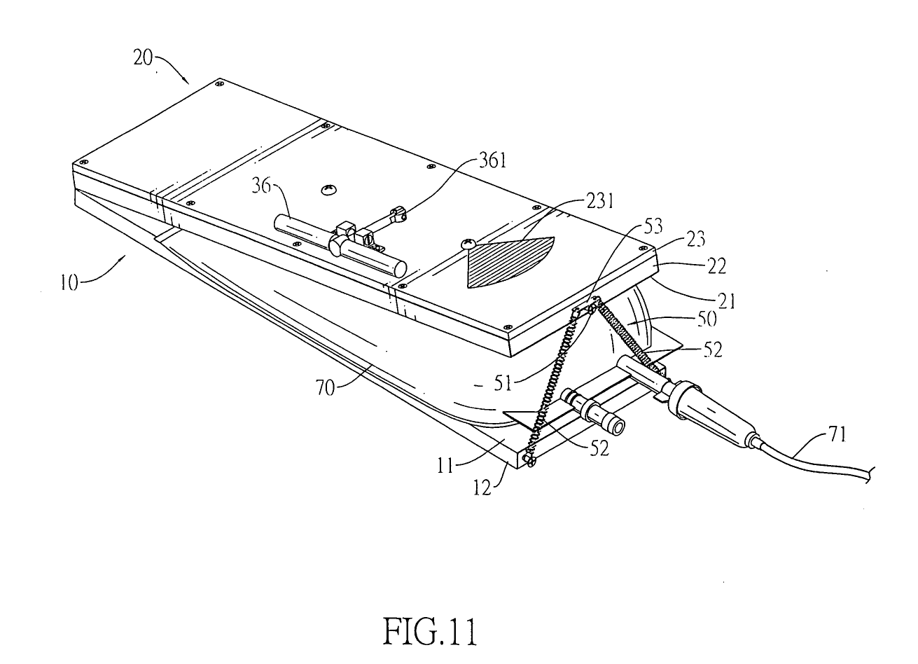

With reference to FIGS. 11, a preferred embodiment of the active squeezing device has an auxiliary pressure assembly providing an additional squeezing force to the drip bag (70) and having a hook (51), two springs (52) and a fastener (53). The hook (51) is mounted on the distal end of the cover (20). The springs (52) are connected to the distal end of the base (10) and each spring (52) has an inside end. The fastener (53) is mounted on the inside ends of the springs (52) and detachably hooks on the hook (51). The fastener (53) hooking on the hook (51) stretches the springs (52) to generate a resilient force for further squeezing the drip bag (70).

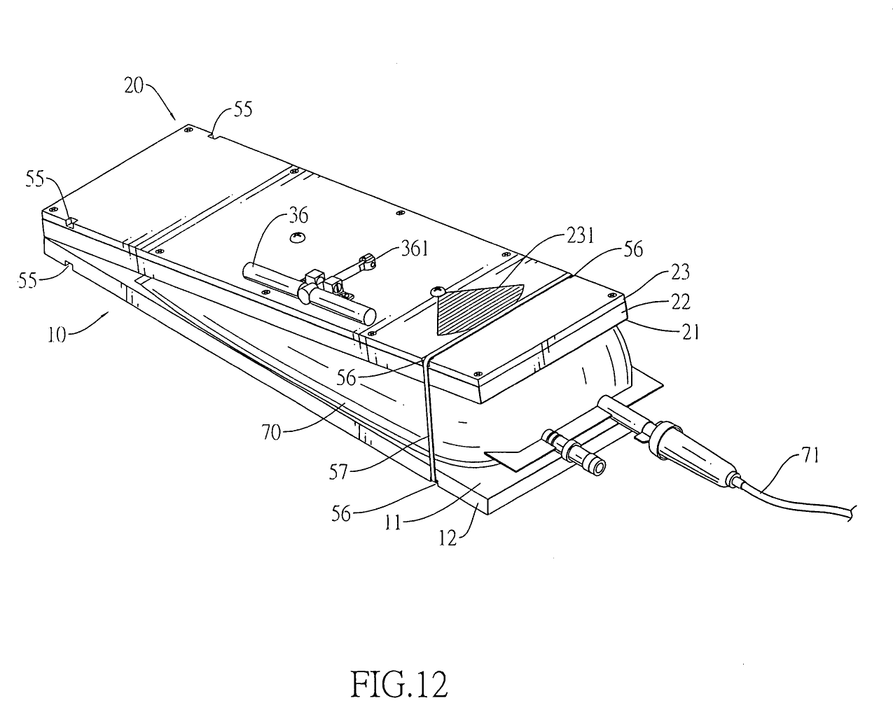

With reference to FIG. 12, a preferred embodiment of the active squeezing device has multiple sets of four notches (55, 56) and an elastic band (57). The sets of notches (55, 56) are defined in the base (10) and the cover (20) and are arranged longitudinally. Two notches (55, 56) of each set are defined in the cover (20) and the other two notches (55, 56) of the set are defined in the base (10). The elastic band (57) is selectively mounted in the notches (55, 56) of one set around the base (10) and the cover (20) to provide a resilient force further squeezing the drip bag (70).

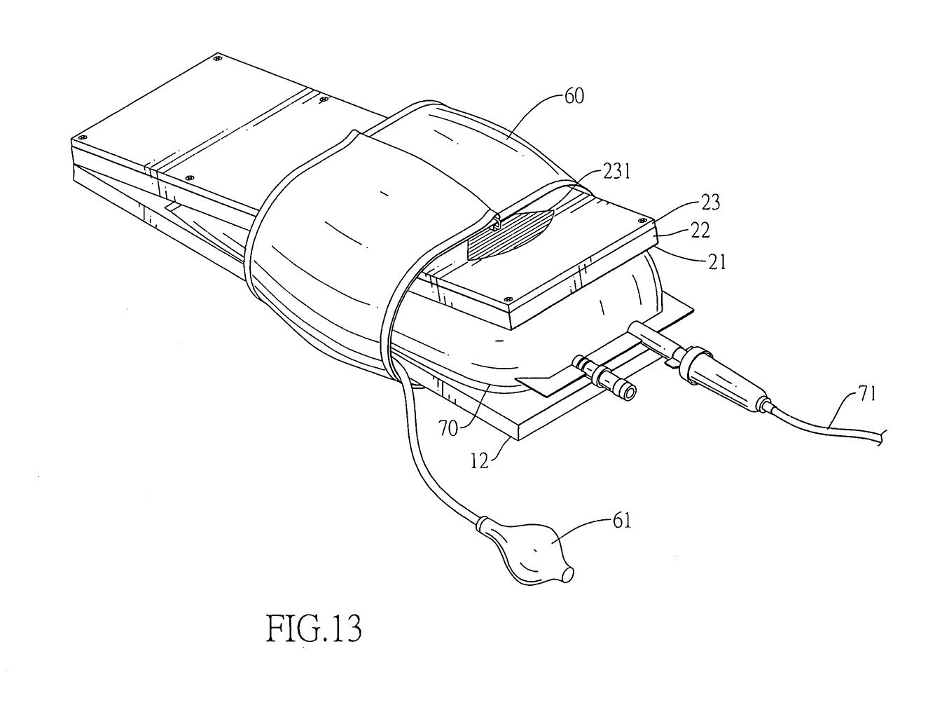

With reference to FIG. 13, the active squeezing device may be used with a blood pressure cuff (60) and an air pump (61). Pumping air into the blood pressure cuff (60) surrounding the base (10) and the cover (20) through the air pump (61) further squeezes the drip bag (70).

The active squeezing device uses the magnetic force to actively transfuse the liquid medicine or blood in the drip bag (70) to a patient without depending on whether the drip bag (70) is located over the patient or not. Therefore, the active squeezing device may be used in a small space such as an internal cavity of a hyperbaric oxygen therapy capsule.

Furthermore, the active squeezing device is light compact as compared with a conventional metal drip stand, is portable and is used without any external power. Therefore, the active squeezing device is not only used in hospitals but also is used in battlefields, which lack power sources and where each person, even a patient, needs to move constantly.

Even though numerous characteristics and advantages of the present invention have been set forth in the foregoing description, together with details of the structure and function of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An active squeezing device for a drip bag comprising:
a drip bag holder having
a base having a proximal end and a distal end; and
a cover mounted pivotally on the base and having a proximal end mounted pivotally on the proximal end of the base and a distal end; and
a magnetic pressure assembly mounted on the drip bag holder and selectively generating an attractive magnetic force to tightly close the cover on the base to be adapted for holding and squeezing a drip bag between the base and the cover or generating a repulsive magnetic force to open the cover to be adapted for releasing the drip bag.

2. The active squeezing device as claimed in claim 1, wherein:
the base has at least one first recess defined in the base;
the cover has at least one first recess defined in the cover; and
the magnetic pressure assembly has
at least one stationary magnetic disk mounted respectively in the at least one first recess in the base and each one of the at least one stationary magnetic disk having multiple south poles and north poles formed on the at least one stationary magnetic disk and arranged alternately;
at least one rotatable magnetic disk rotatably mounted respectively in the at least one first recess in the cover, corresponding to and aligned respectively with the at least one of the stationary magnetic disk and each one of the at least one rotatable magnetic disk having multiple south poles and north poles formed on the at least one stationary magnetic disk and arranged alternately; and
a switch handle mounted pivotally and rotatably on the cover, connected to and capable of rotating the at least one rotatable magnetic disk,
whereby, when the at least rotatable magnetic disk rotates to align the south poles of the at least one of the stationary magnetic disk respectively with the north poles of the at least one rotatable disk to generate an attractive magnetic force, the cover is closed on the base by the attractive magnetic force; and
when the at least one rotatable magnetic disk rotates to align the south poles of the at least one stationary magnetic disk respectively with the south poles of the at least one rotatable magnetic disk to generate a repulsive magnetic force, the cover is opened by the repulsive magnetic force

3. The active squeezing device as claimed in claim 1, wherein:
the cover further has an intermediate recess defined in the cover and communicating with the at least one first recess in the cover;
the magnetic pressure assembly further has at least one follower gear being annular and tightly mounted respectively around the at least one rotatable magnetic disk in the at least first recess in the cover and a driving gear mounted rotatably in the intermediate recess and engaging with the at least one follower gear; and
the switch handle has a pivot member mounted rotatably on the cover and mounted securely on the driving gear and a lever being T-shaped and mounted pivotally on the pivot member.

4. The active squeezing device as claimed in claim 2, wherein the cover further has a positioning assembly mounted on the cover and positioning the lever of the switch handle when the lever lies on the cover.

5. The active squeezing device as claimed in claim 4, wherein:
the base further has a second recess defined in the base;
the cover further has a second recesses defined in the cover; and
the magnetic pressure assembly further has two magnetic elements mounted respectively in the second recesses in the base and the cover, each magnetic has a south pole and a north pole and different poles of the south and north poles of the magnetic elements are aligned with and attract each other.

6. The active squeezing device as claimed in claim 5, wherein:
the magnetic pressure assembly further has a colored disk coaxially mounted respectively on one of the at least on rotatable magnetic disk and having multiple first color segments and second color segments, and the first and second color segments are arranged alternately and correspond to the south and north poles of a corresponding rotatable magnetic disk;
the base further has a base board having two sides, a top surface and a bottom surface in which the at least one first recess and the second recess of the base are defined; and
the cover further has a cover board mounted pivotally on the base board having a top surface in which the at least one first recess and the second recess of the cover are defined, a top panel mounted on the top surface of the cover board and a window attached to the top panel and corresponding to the colored disk.

7. The active squeezing device as claimed in claim 6, wherein the base further has
two sets of multiple slots respectively defined in the sides of the base board and the slots of each set are arranged longitudinally; and
two sets of multiple limits mounted respectively on two sides of the base board and the limits of each set detachably mounted respectively in the slots in a corresponding side, protruding up from the top surface of the base board to block and prevent the cover from pivoting toward the base.

8. The active squeezing device as claimed in claim 7 further comprising an auxiliary pressure assembly having
a hook mounted on the distal end of the cover;
two springs connected to the distal end of the base and each spring has an inside end; and
a fastener mounted on the inside ends of the springs and detachably hooking on the hook.

9. The active squeezing device as claimed in claim 8 further comprising
multiple sets of four notches defined in the base and the cover and arranged longitudinally, two notches of each set defined in the cover and the other two notches of the set defined in the base; and
an elastic band selectively mounted in the notches of one set around the base and the cover.
